(19) United States
(12) Patent Application Publication
(10) Pub. No.: US 2006/0178364 A1
(43) Pub. Date: Aug. 10, 2006

(54) Use of EGFR tyrosinkinase inhibitors for treatment of chronic rhinosinusitis

(75) Inventors: Birgit Jung, Laupheim (DE); Bernd Disse, Mainz (DE); Gerald Pohl, Biberach (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/275,903

(22) Filed: Feb. 2, 2006

(30) Foreign Application Priority Data
Feb. 4, 2005 (DE) 102005005505
Aug. 2, 2005 (DE) 102005036216

(51) Int. Cl. A61K 31/5377; A61K 31/519; A61K 31/517

(52) U.S. Cl. 514/234.2; 514/252.16; 514/252.17; 514/266.4; 514/262.1; 514/264.1

(57) ABSTRACT

The present invention relates to the use of selected EGFR kinase inhibitors, particularly selected quinazolines, quinolines and pyrimido-pyrimidines, for the treatment of nasal polyposis and chronic rhinosinusitis.

The present invention relates to the use of selected EGFR kinase inhibitors, particularly selected quinazolines, quinolines and pyrimido-pyrimidines, for preparing a pharmaceutical composition for the prevention and treatment, particularly for the treatment of nasal polyposis, rhinosinusitis, particularly chronic rhinosinusitis. The invention also relates to the use of tautomers, racemates, stereoisomers, e.g. enantiomers or diastereomers, solvates or hydrates, salts, particularly physiologically acceptable salts with inorganic or organic acids or bases, of the selected EGFR kinase inhibitors.

Patients with chronic rhinosinusitis suffer from an impaired quality of life and this syndrome is often associated with other serious complaints such as for example asthma, eczema and ear infections (otitis media). Nasal polyposis is in many cases the cause of rhinosinusitis and chronic rhinosinusitis. Nasal polyps may for example be caused by allergic rhinitis, acute and chronic rhinitis or by viral or bacterial infections; by irritants, fog and vapours.

Surprisingly the selected EGFR kinase inhibitors lead to a reduction in size (shrinking) of enlarged nasal polyps and are thus suitable for the treatment of nasal polyposis and/or chronic rhinosinusitis as well as in a preventive capacity for preventing relapses after conventional therapy, such as operative polypectomy.

For the purpose according to the invention, for example, the die following compounds 1 selected from among the compounds 1.a to 1.j and 1.1 to 1.101 may be used:
(1.a) 4-[(3-chloro-4-fluoro-phenyl)amino]-7-(2-{4-[(S)-(2-oxo-tetrahydrofuran-5-yl)-carbonyl]-piperazin-1-yl}-ethoxy)-6-[(vinylcarbonyl)amino]-quinazoline,
(1.b) 4-[(3-chloro-4-fluoro-phenyl)amino]-7-[2-((S)-6-methyl-2-oxo-morpholin-4-yl)-ethoxy]-6-[(vinylcarbonyl)amino]-quinazoline,
(1.c) 4-[(3-chloro-4-fluoro-phenyl)amino]-7-[4-((R)-6-methyl-2-oxo-morpholin-4-yl)-butyloxy]-6-[(vinylcarbonyl)amino]-quinazoline,
(1.d) 4-[(3-chloro-4-fluoro-phenyl)amino]-7-[4-((S)-6-methyl-2-oxo-morpholin-4-yl)-butyloxy]-6-[(vinylcarbonyl)amino]-quinazoline,
(1.e) 4-[(3-chloro-4-fluoro-phenyl)amino]-7-[4-(2,2-dimethyl-6-oxo-morpholin-4-yl)-butyloxy]-6-[(vinylcarbonyl)amino]-quinazoline,
(1.f) 4-[(3-chloro-4-fluorophenyl)amino]-6-[(4-{N-[2-(ethoxycarbonyl)-ethyl]-N-[(ethoxycarbonyl)methyl]amino}-1-oxo-2-buten-1-yl)amino]-7-cyclopropyl-methoxy-quinazoline,
(1.g) 4-[(R)-(1-phenyl-ethyl)amino]-6-{[4-(morpholin-4-yl)-1-oxo-2-buten-1-yl]amino}-7-cyclopropylmethoxy-quinazoline,
(1.h) 4-[(3-chloro-4-fluoro-phenyl)amino]-6-({4-[bis-(2-methoxyethyl)-amino]-1-oxo-2-buten-1-yl}amino)-7-cyclopropylmethoxy-quinazoline,
(1.i) 4-[(3-chloro-4-fluoro-phenyl)amino]-6-{[4-((S)-2-methoxymethyl-6-oxo-morpholin-4-yl)-1-oxo-2-buten-1-yl]amino}-7-cyclopropylmethoxy-quinazoline,
(1.j) 4-[(3-chloro-4-fluoro-phenyl)amino]-6-[(4-dimethylamino-cyclohexyl)amino]-pyrimido[5.4-d]pyrimidine,
(1.1) 4-[(3-chloro-4-fluorophenyl)amino]-6-{[4-(morpholin-4-yl)-1-oxo-2-buten-1-yl]-amino}-7-cyclopropylmethoxy-quinazoline,
(1.2) 4-[(3-chloro-4-fluorophenyl)amino]-6-{[4-(N,N-diethylamino)-1-oxo-2-buten-1-yl]-amino}-7-cyclopropylmethoxy-quinazoline,
(1.3) 4-[(3-chloro-4-fluorophenyl)amino]-6-{[4-(N,N-dimethylamino)-1-oxo-2-buten-1-yl]amino}-7-cyclopropylmethoxy-quinazoline,
(1.4) 4-[(R)-(1-phenyl-ethyl)amino]-6-{[4-(morpholin-4-yl)-1-oxo-2-buten-1-yl]amino}-7-cyclopentyloxy-quinazoline,
(1.5) 4-[(3-chloro-4-fluoro-phenyl)amino]-6-{[4-((R)-6-methyl-2-oxo-morpholin-4-yl)-1-oxo-2-buten-1-yl]amino}-7-cyclopropylmethoxy-quinazoline,
(1.6) 4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl )oxy]-quinazoline,
(1.7) 4-[(3-chloro-4-fluoro-phenyl)amino]-6-{[4-((R)-2-methoxymethyl-6-oxo-morpholin-4-yl)-1-oxo-2-buten-1-yl]amino}-7-cyclopropylmethoxy-quinazoline,
(1.8) 4-[(3-chloro-4-fluoro-phenyl)amino]-6-[2-((S)-6-methyl-2-oxo-morpholin-4-yl)-ethoxy]-7-methoxy-quinazoline,
(1.9) 4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline,
(1.10) 4-[(3-chloro-4-fluorophenyl)amino]-6-{[4-(N,N-dimethylamino)-1-oxo-2-buten-1-yl]amino}-7-cyclopentyloxy-quinazoline,
(1.11) 4-[(R)-(1-phenyl-ethyl )amino]-6-{[4-(N,N-bis-(2-methoxy-ethyl )-amino)-1-oxo-2-buten-1-yl]amino}-7-cyclopropylmethoxy-quinazoline,
(1.12) 4-[(R)-(1-phenyl-ethyl )amino]-6-({4-[N-(2-methoxy-ethyl)-N-ethyl-amino]-1-oxo-2-buten-1-yl}amino)-7-cyclopropylmethoxy-quinazoline,
(1.13) 4-[(R)-(1-phenyl-ethyl )amino]-6-({4-[N-(2-methoxy-ethyl)-N-methyl-amino]-1-oxo-2-buten-1-yl}amino)-7-cyclopropylmethoxy-quinazoline,
(1.14) 4-[(R)-(1-phenyl-ethyl )amino]-6-({4-[N-(tetrahydropyran-4-yl )-N-methyl-amino]-1-oxo-2-buten-1-yl}amino)-7-cyclopropylmethoxy-quinazoline,
(1.15) 4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline,
(1.16) 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline,
(1.17) 4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline,
(1.18) 4-[(3-chloro-4-fluorophenyl)amino]-6-{[4-(N-cyclopropyl-N-methyl-amino)-1-oxo-2-buten-1-yl]amino}-7-cyclopentyloxy-quinazoline,
(1.19) 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline,
(1.20) 4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline,
(1.21) 4-[(3-ethynyl-phenyl)amino]-6.7-bis-(2-methoxy-ethoxy)-quinazoline,
(1.22) 4-[(3-chloro-4-fluorophenyl)amino]-7-[3-(morpholin-4-yl)-propyloxy]-6-[(vinyl-carbonyl)amino]-quinazoline,
(1.23) 4-[(R)-(1-phenyl-ethyl)amino]-6-(4-hydroxy-phenyl)-7H-pyrrolo[2.3-d]pyrimidine,
(1.24) 3-cyano-4-[(3-chloro-4-fluorophenyl)amino]-6-{[4-(N,N-dimethylamino)-1-oxo-2-buten-1-yl]amino}-7-ethoxy-quinoline,
(1.25) 3-cyano-4-[(3-chloro-4-(pyridin-2-yl-methoxy)-phenyl)amino]-6-{[4-(N,N-di-methylamino)-1-oxo-2-buten-1-yl]amino}-7-ethoxy-quinoline,
(1.26) 4-{[3-chloro-4-(3-fluoro-benzyloxy)-phenyl]amino}-6-(5-{[(2-methanesulphonyl-ethyl)amino]methyl}-furan-2-yl)quinazoline,
(1.27) 4-[(R)-)1-phenyl-ethyl)amino]-6-{[4-((R)-6-methyl-2-oxo-morpholin-4-yl)-1-oxo-2-buten-1-yl]amino}-7-methoxy-quinazoline,
(1.28) 4-[(3-chloro-4-fluorophenyl)amino]-6-{[4-(morpholin-4-yl)-1-oxo-2-buten-1-yl]-amino}-7-[(tetrahydrofuran-2-yl)methoxy]-quinazoline,
(1.29) 4-[(3-chloro-4-fluorophenyl)amino]-6-({4-[N,N-bis-(2-methoxy-ethyl)-amino]-1-oxo-2-buten-1-yl}amino)-7-[(tetrahydrofuran-2-yl)methoxy]-quinazoline,
(1.30) 4-[(3-ethynyl-phenyl)amino]-6-{[4-(5.5-dimethyl-2-oxo-morpholin-4-yl)-1-oxo-2-buten-1-yl]amino}-quinazoline,
(1.31) 4-[(3-chloro-4-fluoro-phenyl)amino]-6-[2-(2,2-dimethyl-6-oxo-morpholin-4-yl)-ethoxy]-7-methoxy-quinazoline,
(1.32) 4-[(3-chloro-4-fluoro-phenyl)amino]-6-[2-(2,2-dimethyl-6-oxo-morpholin-4-yl)-ethoxy]-7-[(R)-(tetrahydrofuran-2-yl)methoxy]-quinazoline,
(1.33) 4-[(3-chloro-4-fluoro-phenyl)amino]-7-[2-(2,2-dimethyl-6-oxo-morpholin-4-yl)-ethoxy]-6-[(S)-(tetrahydrofuran-2-yl)methoxy]-quinazoline,
(1.34) 4-[(3-chloro-4-fluoro-phenyl)amino]-6-{2-[4-(2-oxo-morpholin-4-yl)-piperidin-1-yl]-ethoxy}-7-methoxy-quinazoline,
(1.35) 4-[(3-chloro-4-fluoro-phenyl)amino]-6-[1-(tert-butyloxycarbonyl)-piperidin-4-yloxy]-7-methoxy-quinazoline,
(1.36) 4-[(3-chloro-4-fluoro-phenyl)amino]-6-(trans-4-amino-cyclohexan-1-yloxy)-7-methoxy-quinazoline,
(1.37) 4-[(3-chloro-4-fluoro-phenyl)amino]-6-(trans-4-methanesulphonylamino-cyclohexan-1-yloxy)-7-methoxy-quinazoline,
(1.38) 4-[(3-chloro-4-fluoro-phenyl)amino]-6-(tetrahydropyran-3-yloxy)-7-methoxy-quinazoline,
(1.39) 4-[(3-chloro-4-fluoro-phenyl)amino]-6-)1-methyl-piperidin-4-yloxy)-7-methoxy-quinazoline,
(1.40) 4-[(3-chloro-4-fluoro-phenyl)amino]-6-{l -[(morpholin-4-yl)carbonyl]-piperidin-4-yloxy}-7-methoxy-quinazoline,
(1.41) 4-[(3-chloro-4-fluoro-phenyl)amino]-6-{l -[(methoxymethyl)carbonyl]-piperidin-4-yloxy}-7-methoxy-quinazoline,
(1.42) 4-[(3-chloro-4-fluoro-phenyl)amino]-6-(piperidin-3-yloxy)-7-methoxy-quinazoline,
(1.43) 4-[(3-chloro-4-fluoro-phenyl)amino]-6-[1-(2-acetylamino-ethyl)-piperidin-4-yloxy]-7-methoxy-quinazoline,
(1.44) 4-[(3-chloro-4-fluoro-phenyl)amino]-6-(tetrahydropyran-4-yloxy)-7-ethoxy-quinazoline,
(1.45) 4-[(3-chloro-4-fluoro-phenyl)amino]-6-((S)-tetrahydrofuran-3-yloxy)-7-hydroxy-quinazoline,
(1.46) 4-[(3-chloro-4-fluoro-phenyl)amino]-6-(tetrahydropyran-4-yloxy)-7-(2-methoxy-ethoxy)-quinazoline,
(1.47) 4-[(3-chloro-4-fluoro-phenyl)amino]-6-{trans-4-[(dimethylamino)sulphonylamino]-cyclohexan-1-yloxy}-7-methoxy-quinazoline,
(1.48) 4-[(3-chloro-4-fluoro-phenyl)amino]-6-{trans-4-[(morpholin-4-yl)carbonylamino]-cyclohexan-1-yloxy}-7-methoxy-quinazoline,
(1.49) 4-[(3-chloro-4-fluoro-phenyl)amino]-6-{trans-4-[(morpholin-4-yl)sulphonylamino]-cyclohexan-1-yloxy}-7-methoxy-quinazoline,
(1.50) 4-[(3-chloro-4-fluoro-phenyl)amino]-6-(tetrahydropyran-4-yloxy)-7-(2-acetylamino-ethoxy)-quinazoline,
(1.51) 4-[(3-chloro-4-fluoro-phenyl)amino]-6-(tetrahydropyran-4-yloxy)-7-(2-methanesulphonylamino-ethoxy)-quinazoline,
(1.52) 4-[(3-chloro-4-fluoro-phenyl)amino]-6-{1-[(piperidin-1yl)carbonyl]-piperidin-4-yloxy}-7-methoxy-quinazoline,
(1.53) 4-[(3-chloro-4-fluoro-phenyl)amino]-6-(1-aminocarbonylmethyl-piperidin-4-yl-oxy)-7-methoxy-quinazoline,
(1.54) 4-[(3-chloro-4-fluoro-phenyl)amino]-6-(cis-4-{N-[(tetrahydropyran-4-yl)carbonyl]-N-methyl-amino}-cyclohexan-1-yloxy)-7-methoxy-quinazoline,
(1.55) 4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline,
(1.56) 4-[(3-chloro-4-fluoro-phenyl)amino]-6-(cis-4-{N-[(morpholin-4-yl)sulphonyl]-N-methyl-amino}-cyclohexan-1-yloxy)-7-methoxy-quinazoline,
(1.57) ⁴-[(³-chloro-4-fluoro-phenyl)amino]-6-(trans-4-ethansulphonylamino-cyclohexan-1-yloxy)-7-methoxy-quinazoline,
(1.58) 4-[(3-chloro-4-fluoro-phenyl)amino]-6-(1-methanesulphonyl-piperidin-4-yloxy)-7-ethoxy-quinazoline,
(1.59) 4-[(3-chloro-4-fluoro-phenyl)amino]-6-(1-methanesulphonyl-piperidin-4-yloxy)-7-(2-methoxy-ethoxy)-quinazoline,
(1.60) 4-[(3-chloro-4-fluoro-phenyl)amino]-6-[l-(2-methoxy-acetyl)-piperidin-4-yloxy]-7-(2-methoxy-ethoxy)-quinazoline,
(1.61) 4-[(3-chloro-4-fluoro-phenyl)amino]-6-(cis-4-acetylamino-cyclohexan-1-yloxy)-7-methoxy-quinazoline,
(1.62) 4-[(3-ethynyl-phenyl)amino]-6-[l -(tert.-butyloxycarbonyl)-piperidin-4-yloxy]-7-methoxy-quinazoline,
(1.63) 4-[(3-ethynyl-phenyl)amino]-6-(tetrahydropyran-4-yloxy]-7-methoxy-quinazoline,
(1.64) 4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline,
(1.65) 4-[(3-chloro-4-fluoro-phenyl)amino]-6-(cis-4-{N-[(4-methyl-piperazin-1-yl)-carbonyl]-N-methyl-amino}-cyclohexan-1-yloxy)-7-methoxy-quinazoline,
(1.66) 4-[(3-chloro-4-fluoro-phenyl)amino]-6-{cis-4-[(morpholin-4-yl)carbonylamino]-cyclohexan-1-yloxy}-7-methoxy-quinazoline,
(1.67) 4-[(3-chloro-4-fluoro-phenyl)amino]-6-{l -[2-(2-oxopyrrolidin-1-yl)ethyl]-piperidin-4-yloxy}-7-methoxy-quinazoline,
(1.68) 4-[(3-chloro-4-fluoro-phenyl)amino]-6-{l -[(morpholin-4-yl)carbonyl]-piperidin-4-yloxy}-7-(2-methoxy-ethoxy)-quinazoline,
(1.69) 4-[(3-ethynyl-phenyl)amino]-6-(1-acetyl-piperidin-4-yloxy)-7-methoxy-quinazoline,
(1.70) 4-[(3-ethynyl-phenyl)amino]-6-(I-methyl-piperidin-4-yloxy)-7-methoxy-quinazoline,
(1.71) 4-[(3-ethynyl-phenyl)amino]-6-)1-methanesulphonyl-piperidin-4-yloxy)-7-methoxy-quinazoline,
(1.72) 4-[(3-chloro-4-fluoro-phenyl)amino]-6-)1-methyl-piperidin-4-yloxy)-7(2-methoxy-ethoxy)-quinazoline,
(1.73) 4-[(3-chloro-4-fluoro-phenyl)amino]-6-(I-isopropyloxycarbonyl-piperidin-4-yloxy)-7-methoxy-quinazoline,
(1.74) 4-[(3-chloro-4-fluoro-phenyl)amino]-6-(cis-4-methylamino-cyclohexan-1-yloxy)-7-methoxy-quinazoline,
(1.75) 4-[(3-chloro-4-fluoro-phenyl)amino]-6-{cis-4-[N-(2-methoxy-acetyl)-N-methyl-amino]-cyclohexan-1-yloxy}-7-methoxy-quinazoline,
(1.76) 4-[(3-ethynyl-phenyl)amino]-6-(piperidin-4-yloxy)-7-methoxy-quinazoline,
(1.77) 4-[(3-ethynyl-phenyl)amino]-6-[1-(2-methoxy-acetyl)-piperidin-4-yloxy]-7-methoxy-quinazoline,
(1.78) 4-[(3-ethynyl-phenyl)amino]-6-{1-[(morpholin-4-yl)carbonyl]-piperidin-4-yloxy}-7-methoxy-quinazoline,
(1.79) 4-[(3-chloro-4-fluoro-phenyl)amino]-6-{1-[(cis-2.6-dimethyl-morpholin-4-yl)carbonyl]-piperidin-4-yloxy}-7-methoxy-quinazoline,
(1.80) 4-[(3-chloro-4-fluoro-phenyl)amino]-6-{1-[(2-methyl-morpholin-4-yl)carbonyl]-piperidin-4-yloxy}-7-methoxy-quinazoline,
(1.81) 4-[(3-chloro-4-fluoro-phenyl)amino]-6-{1-[(S,S)-(2-oxa-5-aza-bicyclo[2,2,1]hept-5-yl)carbonyl]-piperidin-4-yloxy}-7-methoxy-quinazoline,
(1.82) 4-[(3-chloro-4-fluoro-phenyl)amino]-6-{l -[(N-methyl-N-2-methoxyethyl-amino)-carbonyl]-piperidin-4-yloxy}-7-methoxy-quinazoline,
(1.83) 4-[(3-chloro-4-fluoro-phenyl)amino]-6-)1-ethyl-piperidin-4-yloxy)-7-methoxy-quinazoline,
(1.84) 4-[(3-chloro-4-fluoro-phenyl)amino]-6-{l -[(2-methoxyethyl)carbonyl]-piperidin-4-yloxy}-7-methoxy-quinazoline,
(1.85) 4-[(3-chloro-4-fluoro-phenyl)amino]-6-{l-[(3-methoxypropyl-amino)-carbonyl]-piperidin-4-yloxy}-7-methoxy-quinazoline,
(1.86) 4-[(3-chloro-4-fluoro-phenyl)amino]-6-[cis-4-(N-methanesulphonyl-N-methyl-amino)-cyclohexan-1-yloxy]-7-methoxy-quinazoline,
(1.87) 4-[(3-chloro-4-fluoro-phenyl)amino]-6-[cis-4-(N-acetyl-N-methyl-amino)-cyclohexan-1-yloxy]-7-methoxy-quinazoline,
(1.88) 4-[(3-chloro-4-fluoro-phenyl)amino]-6-(trans-4-methylamino-cyclohexan-1-yloxy)-7-methoxy-quinazoline,
(1.89) 4-[(3-chloro-4-fluoro-phenyl)amino]-6-[trans-4-(N-methanesulphonyl-N-methyl-amino)-cyclohexan-1-yloxy]-7-methoxy-quinazoline,
(1.90) 4-[(3-chloro-4-fluoro-phenyl)amino]-6-(trans-4-dimethylamino-cyclohexan-1-yloxy)-7-methoxy-quinazoline,
(1.91) 4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline,
(1.92) 4-[(3-chloro-4-fluoro-phenyl)amino]-6-{l -[2-(2-oxo-3-methyl-imidazolidin-1-yl)-ethyl]-piperidin-4-yloxy}-7-methoxy-quinazoline,
(1.93) 4-[(3-chloro-4-fluoro-phenyl)amino]-6-{1-[2-(2-oxo-hexahydropyrimidin-1-yl)-ethyl]-piperidin-4-yloxy}-7-methoxy-quinazoline,
(1.94) 4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline,
(1.95) 4-[(3-chloro-4-fluoro-phenyl)amino]-6-(1-methanesulphonyl-piperidin-4-yloxy)-7-methoxy-quinazoline,
(1.96) 4-[(3-chloro-4-fluoro-phenyl)amino]-6-(1-cyano-piperidin-4-yloxy)-7-methoxy-quinazoline,
(1.97) 4-[(3-chloro-4-fluoro-phenyl)amino]-6-(tetrahydropyran-4-yloxy)-7-methoxy-quinazoline,
(1.98) 4-[(3-chloro-4-fluoro-phenyl)amino]-6-(1-methylcarbonyl-piperidin-4-yloxy)-7-methoxy-quinazoline,
(1.99) 4-[(3-chloro-4-fluoro-phenyl)amino]-6-(1-dimethylaminoacetyl-piperidin-4-yloxy)-7-methoxy-quinazoline,
(1.100) 4-[(3-chloro-4-fluoro-phenyl)amino]-6-{1-[(dimethylamino)carbonylmethyl]-piperidin-4-yloxy}-7-methoxy-quinazoline,
(1.101) 4-[(3-chloro-4-fluoro-phenyl)amino]-6-(1-methanesulphonyl-piperidin-4-yloxy)-quinazoline,
or the salts thereof.

The compounds are known per se from the prior art, the preparation thereof is described for example in the following publications:

WO 96/30347; WO 97/02266; WO 97/32880, WO 99/35146; WO 00/31048; WO 00/51991, WO 00/78735; WO 01/34574; WO 01/61816; WO 01/77104; WO 02/18351; WO 02/18370, WO 02/18372; WO 02/18373; WO 02/18375, WO 02/18376; WO 02/50043; WO 03/082290; Cancer Research 2004, 64:11 (3958-3965); Am J Health-Syst Pharm 2000, 57(15), 2063-2076; Clinical Therapeutics 1999, 21(2), 309-318; WO 98/50433; and WO 95/20045.

In all the embodiments or aspects of the invention the EGFR kinase inhibitors 1.1 to 1.101 are preferred, particularly the EGFR kinase inhibitors 1.1, 1.4, 1.6, 1.8, 1.9, 1.14, 1.17, 1.19, 1.21, 1.23, 1.24, 1.27, 1.28, 1.30, 1.34, 1.35, 1.37, 1.38, 1.40, 1.42, 1.43, 1.44, 1.48, 1.52, 1.55, 1.57, 1.59, 1.60, 1.63, 1.64, 1.66, 1.67, 1.69, 1.70, 1.71, 1.72, 1.78, 1.82, 1.83, 1.84, 1.88, 1.90, 1.91, 1.94 and 1.95

or the salts thereof.

Physiologically acceptable acid addition salts of the compounds of group 1 are for example the hydrochloride, hydrobromide, hydriodide, hydrosulphate, hydrophosphate, hydromethanesulphonate, hydronitrate, hydromaleinate, hydroacetate, hydrobenzoate, hydrocitrate, hydrofumarate, hydrotartrate, hydrolactate, hydroxalate, hydrosuccinate, hydrobenzoate and hydro-p-toluenesulphonate.

The present invention further relates to a method of preventing and/or treating chronic rhinosinusitis, nasal polyposis and chronic rhinosinusitis with nasal polyposis, preferably chronic rhinosinusitis with nasal polyposis, comprising administering an effective amount of one or more of the above-mentioned compounds (1.a) to (1.j) or (1.1) to (1.101) or optionally one of the physiologically acceptable salts thereof to a patient requiring such treatment. The compounds are administered in a suitable pharmaceutical form, preferably in a preparation suitable for nasal application, for example in the form of a solution, a suspension, an aerosol or a powder. They are preferably used in monotherapy and the process is also preferably a treatment process.

By the term “prevention” or “preventative treatment” is meant a treatment for the purpose of reducing the risk of developing one of the above-mentioned conditions, particularly in patients at increased risk of these indications, where there is a pre-existing history or corresponding anamnesis, e.g. in patients who have already undergone conventional therapy, e.g. operative polypectomy. The success of preventative treatment by reducing the occurrence of the indications in question in a corresponding population of patients at risk is statistically provable, by comparison with a population of patients at risk without preventative treatment.

By the term “treatment” is meant a therapeutic treatment of patients with manifest, acute or chronic indications, including on the one hand symptomatic (palliative) treatment to alleviate the symptoms of the disease and on the other hand causal or curative treatment of the indication, with the aim of bringing the pathological condition to an end, reducing the severity of the pathological condition or delaying the progression of the pathological condition, depending on the nature or gravity of the indication.

In the process according to the invention the compounds mentioned above are used in doses of 0.001-500 mg per application, preferably 0.01-50 mg, particularly preferably 0.02 to 10 mg, conveniently being given 1 to 3 times a day.

The nasal application of the active substances may be effected for example by administering nasal drops or, using known dosing systems, in the form of a nasal spray (solution or suspension), from aqueous solutions or suspensions as an aerosol or by means of powders for intranasal deposition.

Some of the active substances 1.a -1.j and 1.1-1.101 contain a hydrolysis-sensitive ester or lactone group. Conveniently, substantially anhydrous formulations and preparations are selected for these compounds. In these cases the compounds are preferably administered by nasal route as powders for intranasal deposition.

The powder formulations which may be used for nasal application within the scope of the use according to the invention may contain the active substance or the active substance combination either on their own or in admixture with suitable, preferably physiologically acceptable excipients.

The excipients used in nasal powders are carriers (which transport a finely micronised active substance), gelling agents (which slow down the removal of the active substance from the nasal cavity), fillers (for bulking up low-dose active substances to a manageable volume) or enhancers (which improve the absorption of the active substance), while an excipient may also perform a number of functions at the same time.

Examples of carriers include the following excipients, including mixtures thereof (some of these carriers simultaneously acting as enhancers):

Cellulose or the ester and ether derivatives thereof: microcrystalline cellulose, microfine cellulose, methylcellulose, ethylcellulose, hydroxymethylcellulose, hydroxyethylcellulose, hydroxypropylcellulose, hydroxypropylmethylcellulose, sodium carboxymethylcellulose;

disaccharides such as lactose or lactose monohydrate, sucrose and maltose;

starch: starch microparticles, crosslinked starch, (crosslinked) starch derivatives;

oligo- and polysaccharides such as dextran microparticles (Sephadex®); cyclodextrins, for example β-cyclodextrin or dimethyl-β-cyclodextrin; (crosslinked) polyvinylpyrollidone; gelatine, chitin, chitosan, gum tragacanth, polyacrylate, alginic acid, polyethyleneglycols (molecular weight approx. 1000-8000 Da).

Depending on their properties the following excipients, including the mixtures thereof, may be used as enhancers for the various formulations, for example:

surfactants: non-ionic, ionic and amphoteric surfactants, for example polyoxyethylene-9-laurylether, Poloxamer 407, Brij 35, Brij 96, polysorbate 80, soybean-derived sterylglucoside;

bile salts or derivatives, for example Na-deoxycholate, Na-glycocholate, sodium-tauro-24,25-dihydrofusidate (sodium-tauro-24,25-dihydrofusidate [STDHF]);

fatty acid derivatives, for example oleic acid, lauroylcarnitine, acylcarnitine, palmitoyl-DL-carnitine;

phospholipids, for example didecanoyl-L-alpha-phosphatidylcholine, dimyristoyl-phosphatidylglycerol, lysophosphatidylcholine, semisynthetic lysophosphatidylcholine variants, dipalmitoyl-phosphatidylcholine;

cyclodextrins and derivatives, for example α-, β- or γ-cyclodextrin, dimethyl-β-cyclodextri n, hydroxypropyl-o-cyclodextrin;

chitosan and derivatives, for example poly-L-arginine-chitosan, hyaluronan chitosan;

diethylaminoethyl-dextran, glycyrrhetinic acid, EDTA, hyaluronic acid ester.

Examples of particular excipients include:

mucolytics, for example N-acetylcysteine or ambroxol, for reducing the viscosity of the mucus, thereby improving the diffusion and absorption of the active substance (described in WO 04078211);

Other excipients in question are:

human serum albumin, polyalcohols (e.g. mannitol, sorbitolol, xylitol), trehalose, amino acids, monosaccharides (e.g. glucose or arabinose), casein, salts (e.g. sodium chloride, calcium carbonate) or mixtures of these excipients with one another.

The particle sizes of the active substance and of any excipients used suitable for nasal deposition are described in the prior art, as are suitable methods of achieving the required particle size distributions, e.g. by grinding, micronising or spray-drying. Purely by way of example the following documents are mentioned in this context: EP 1124544; US 2005019411; EP 1 036 562; WO 95/05805; Farmacopea Europea, III Edition 1997, Paragraph 2.9.18., page 143. Details of particle sizes or particle size distributions refer, unless otherwise stated, to the aerodynamic diameter, determined, for particle sizes of 10 μm or less, by means of a cascade impactor, and for particle sizes of more than 10 μm by means of laser diffraction. Both methods of measurement are described in the prior art. Particle sizes suitable for nasal deposition (aerodynamic diameter) are in the range between about 10 and 200 μm. With particle sizes of less than 10 μm, particularly less than about 5 μm, the particles are inhalable and enter the lungs.

In suitable nasal powders the excipients have an average particle size of up to 350 μm, preferably between 10 and 150 μm, particularly preferably between 15 and 80 μm. The active substance is added to the excipient in an average particle size of 0.1 to 200 μm, preferably 5 to 25 μm, particularly preferably 10 to 25 μm. Active substance with an average particle size of 0.1 to 5 μm is formulated with a carrier which has a particle size spectrum suitable for nasal deposition. Active substance with an average particle size of 5 to 10 μm is preferably, but not necessarily, formulated with a carrier which has a particle size spectrum suitable for nasal deposition. The powders may be applied, for example, from capsules using suitable inhalers described in the prior art.

The aerosols containing propellant gas which may be used for the purposes of the invention may contain the active substance or combination of active substances dissolved in the propellant gas or in dispersed form. The propellant gases which may be used to prepare the aerosols are known from the prior art. Suitable propellant gases are selected from among hydrocarbons such as n-propane, n-butane or isobutane and halohydrocarbons such as preferably fluorinated derivatives of methane, ethane, propane, butane, cyclopropane or cyclobutane. The above-mentioned propellant gases may be used on their own or mixed together. Particularly preferred propellant gases are halogenated alkane derivatives selected from TG134a (1,1,1,2-tetrafluoroethane), TG227 (1,1,1,2,3,3,3-heptafluoropropane) and mixtures thereof.

The propellant-driven inhalation aerosols may also contain other ingredients such as co-solvents, stabilisers, surfactants, antioxidants, lubricants and pH adjusters. All these ingredients are known in the art.

If the active substance or combination of active substances according to the invention is administered in the form of propellant-free solutions or suspensions it is possible to use aqueous, oily or alcoholic solutions, for example ethanolic solutions, as the solvent or suspension agent. The solvent may be water on its own or a mixture of water and ethanol. Similarly, sterile aqueous solutions of the corresponding pharmaceutically acceptable salts may be used. Furthermore solutions or suspensions of the active substances in aqueous propyleneglycol, in sesame or groundnut oil are possible. Aqueous solutions should be suitably buffered if necessary and the liquid diluent should be made isotonic for example with sufficient salt or glucose. Depending on the optimum stability and compatibility the solutions or suspensions containing the active substance or combination of active substances may be adjusted to a pH of 2 to 9, preferably 2 to 7, particularly 2 to 5 with suitable physiologically acceptable acids or bases. This pH may be adjusted using acids selected from inorganic or organic acids. Examples of particularly suitable inorganic acids are hydrochloric acid, hydrobromic acid, nitric acid, sulphuric acid and/or phosphoric acid. Examples of particularly suitable organic acids are: ascorbic acid, citric acid, malic acid, tartaric acid, maleic acid, succinic acid, fumaric acid, acetic acid, formic acid and/or propionic acid and others. Preferred inorganic acids are hydrochloric acid, sulphuric acid. Of the organic acids ascorbic acid, fumaric acid and citric acid are preferred. If desired, mixtures of the above acids may also be used, particularly in the case of acids which have other properties in addition to their acidifying qualities, e.g. as antioxidants or complexing agents, such as citric acid or ascorbic acid, for example. Suitable bases include for example sodium hydroxide, potassium hydroxide, arginine, cyclohexylamine, ethanolamine, diethanolamine and triethanolamine.

As already mentioned hereinbefore, the compounds 1 and the salts thereof have valuable properties and may be used in particular for the treatment of nasal polyposis, rhinosinusitis, preferably chronic rhinosinusitis or chronic rhinosinusitis with nasal polyposis.

For example the compounds

1.a. 1.b. 1.c. 1.d. 1.e. 1.5. 1.6, 1.7. 1.8, 1.27, 1.30, 1.31, 1.32, 1.33, 1.34 and 1.94.

were prepared as nasal formulations for topical administration.

### EXAMPLES OF FORMULATIONS

The following Examples serve to illustrate the invention without limiting the subject of the invention. Examples of pharmaceutical formulations of compounds of group 1 may also be found in the prior art, for example the specifications mentioned hereinbefore.

The formulations described in Examples 1 to 5 and 7 contain any desired active substance selected from the group 1. The formulation of Example 6 contains an active substance selected from the group 1, which does not have a hydrolysis-sensitive group, such as an ester or lactone group, for example.

A) Powders for Intranasal Application:

### Example 1

| constituents | μg per capsule |
|---|---|
| active substance | 150 |
| lactose | 12300 |
| Total | 12450 |

### Example 2

| constituents | μg per capsule |
|---|---|
| active substance | 1500 |
| lactose | 12200 |
| Total | 13700 |

### Example 3

| constituents | μg per capsule |
|---|---|
| active substance | 5000 |
| lactose | 15000 |
| Total | 20000 |

### Example 4

| constituents | μg per capsule |
|---|---|
| active substance | 7500 |
| lactose | 20000 |
| Total | 27500 |

### Example 5

| constituents | μg per capsule |
|---|---|
| active substance | 7500 |
| Total | 7500 |

### General Information Relating to Examples 1 to 5

The active substance and the excipient (if used) (lactose monohydrate) are, if necessary, micronised in each case by conventional methods, optionally spheronised and screened, and optionally then mixed in the desired mixing ratio. For the active substance an average particle size of between 5 and 200 μm (aerodynamic diameter) is selected, for example in the range between 10 to 25 μm, while the average particle size of the excipient is conveniently selected in the range from 10 to 350 μm, for example between 15 and 80 μm.

B) Solutions for Intranasal Application:

### Example 6

Nasal spray containing 1 mg active substance
Composition:

| active substance | 1.0 | mg (based on the free base) |
|---|---|---|
| sodium chloride | q.s. to make it isotonic | |
| benzalkonium chloride | 0.025 | mg |
| disodium edetate | 0.05 | mg |
| water purified ad | 0.1 | ml |

Method of Preparation:

The active substance in the form of a physiologically acceptable salt and the excipients are dissolved in water and transferred into a corresponding container.

### Example 7

Nasal spray with 1 mg active substance
Composition:

| | |
|---|---|
| active substance | 1.0 mg (based on the free base) |
| sesame oil ad | 0.1 ml |

Method of Preparation:

The active substance is dissolved in sesame oil and transferred into a corresponding container.

What is claimed is:

1. A method for treating chronic rhinosinusitis, nasal polyposis or chronic rhinosinusitis with nasal polyposis which comprises administering a compound selected from the group consisting of:
(1.a) 4-[(3-chloro-4-fluoro-phenyl)amino]-7-(2-{4-[(S)-(2-oxo-tetrahydrofuran-5-yl)-carbonyl]-piperazin-1-yl}-ethoxy)-6-[(vinylcarbonyl)amino]-quinazoline,
(1.b) 4-[(3-chloro-4-fluoro-phenyl)amino]-7-[2-((S)-6-methyl-2-oxo-morpholin-4-yl)-ethoxy]-6-[(vinylcarbonyl)amino]-quinazoline,
(1.c) 4-[(3-chloro-4-fluoro-phenyl)amino]-7-[4-((R)-6-methyl-2-oxo-morpholin-4-yl)-butyloxy]-6-[(vinylcarbonyl)amino]-quinazoline,
(1.d) 4-[(3-chloro-4-fluoro-phenyl)amino]-7-[4-((S)-6-methyl-2-oxo-morpholin-4-yl)-butyloxy]-6-[(vinylcarbonyl)amino]-quinazoline,
(1.e) 4-[(3-chloro-4-fluoro-phenyl)amino]-7-[4-(2,2-dimethyl-6-oxo-morpholin-4-yl)-butyloxy]-6-[(vinylcarbonyl)amino]-quinazoline,
(1.f) 4-[(3-chloro-4-fluorophenyl)amino]-6-[(4-{N-[2-(ethoxycarbonyl)-ethyl]-N-[(ethoxycarbonyl)methyl]amino}-1-oxo-2-buten-1-yl)amino]-7-cyclopropyl-methoxy-quinazoline,
(1.g) 4-[(R)-(1-phenyl-ethyl)amino]-6-{[4-(morpholin-4-yl)-1-oxo-2-buten-1-yl]amino}-7-cyclopropylmethoxy-quinazoline,
(1.h) 4-[(3-chloro-4-fluoro-phenyl)amino]-6-({4-[bis-(2-methoxyethyl)-amino]-1-oxo-2-buten-1-yl}amino)-7-cyclopropylmethoxy-quinazoline,
(1.i) 4-[(3-chloro-4-fluoro-phenyl)amino]-6-{[4-((S)-2-methoxymethyl-6-oxo-morpholin-4-yl)-1-oxo-2-buten-1-yl]amino}-7-cyclopropylmethoxy-quinazoline,
(1.j) 4-[(3-chloro-4-fluoro-phenyl)amino]-6-[(4-dimethylamino-cyclohexyl)amino]-pyrimido[5.4-d]pyrimidine,
(1.1) 4-[(3-chloro-4-fluorophenyl)amino]-6-{[4-(morpholin-4-yl)-1-oxo-2-buten-1-yl]-amino}-7-cyclopropylmethoxy-quinazoline,
(1.2) 4-[(3-chloro-4-fluorophenyl)amino]-6-{[4-(N,N-diethylamino)-1-oxo-2-buten-1-yl]-amino}-7-cyclopropylmethoxy-quinazoline,
(1.3) 4-[(3-chloro-4-fluorophenyl)amino]-6-{[4-(N,N-dimethylamino)-1-oxo-2-buten-1-yl]amino}-7-cyclopropylmethoxy-quinazoline,
(1.4) 4-[(R)-(1-phenyl-ethyl)amino]-6-{[4-(morpholin-4-yl)-1-oxo-2-buten-1-yl]amino}-7-cyclopentyloxy-quinazoline,
(1.5) 4-[(3-chloro-4-fluoro-phenyl)amino]-6-{[4-((R)-6-methyl-2-oxo-morpholin-4-yl)-1-oxo-2-buten-1-yl]amino}-7-cyclopropylmethoxy-quinazoline,
(1.6) 4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline,
(1.7) 4-[(3-chloro-4-fluoro-phenyl)amino]-6-{[4-((R)-2-methoxymethyl-6-oxo-morpholin-4-yl)-1-oxo-2-buten-1-yl]amino}-7-cyclopropylmethoxy-quinazoline,
(1.8) 4-[(3-chloro-4-fluoro-phenyl)amino]-6-[2-((S)-6-methyl-2-oxo-morpholin-4-yl)-ethoxy]-7-methoxy-quinazoline,
(1.9) 4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline,
(1.10) 4-[(3-chloro-4-fluorophenyl)amino]-6-{[4-(N,N-dimethylamino)-1-oxo-2-buten-1-yl]amino}-7-cyclopentyloxy-quinazoline,
(1.11) 4-[(R)-(1-phenyl-ethyl)amino]-6-{[4-(N,N-bis-(2-methoxy-ethyl)-amino)-1-oxo-2-buten-1-yl]amino}-7-cyclopropylmethoxy-quinazoline,
(1.12) 4-[(R)-(1-phenyl-ethyl)amino]-6-({4-[N-(2-methoxy-ethyl)-N-ethyl-amino]-1-oxo-2-buten-1-yl}amino)-7-cyclopropylmethoxy-quinazoline,
(1.13) 4-[(R)-(1-phenyl-ethyl)amino]-6-({4-[N-(2-methoxy-ethyl)-N-methyl-amino]-1-oxo-2-buten-1-yl}amino)-7-cyclopropylmethoxy-quinazoline,
(1.14) 4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline,
(1.15) 4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline,
(1.16) 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline,
(1.17) 4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline,
(1.18) 4-[(3-chloro-4-fluorophenyl)amino]-6-{[4-(N-cyclopropyl-N-methyl-amino)-1-oxo-2-buten-1-yl]amino}-7-cyclopentyloxy-quinazoline,
(1.19) 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline,
(1.20) 4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline,
(1.21) 4-[(3-ethynyl-phenyl)amino]-6.7-bis-(2-methoxy-ethoxy)-quinazoline,
(1.22) 4-[(3-chloro-4-fluorophenyl)amino]-7-[3-(morpholin-4-yl)-propyloxy]-6-[(vinyl-carbonyl)amino]-quinazoline,
(1.23) 4-[(R)-(1-phenyl-ethyl)amino]-6-(4-hydroxy-phenyl)-7H-pyrrolo[2.3-d]pyrimidine,
(1.24) 3-cyano-4-[(3-chloro-4-fluorophenyl)amino]-6-{[4-(N,N-dimethylamino)-1-oxo-2-buten-1-yl]amino}-7-ethoxy-quinoline,
(1.25) 3-cyano-4-[(3-chloro-4-(pyridin-2-yl-methoxy)-phenyl)amino]-6-{[4-(N,N-di-methylamino)-1-oxo-2-buten-1-yl]amino}-7-ethoxy-quinoline,
(1.26) 4-{[3-chloro-4-(3-fluoro-benzyloxy)-phenyl]amino}-6-(5-{[(2-methanesulphonyl-ethyl)amino]methyl}-furan-2-yl)quinazoline,
(1.27) 4-[(R)-)1-phenyl-ethyl)amino]-6-{[4-((R)-6-methyl-2-oxo-morpholin-4-yl)-1-oxo-2-buten-1-yl]amino}-7-methoxy-quinazoline,
(1.28) 4-[(3-chloro-4-fluorophenyl)amino]-6-{[4-(morpholin-4-yl)-1-oxo-2-buten-1-yl]-amino}-7-[(tetrahydrofuran-2-yl)methoxy]-quinazoline,
(1.29) 4-[(3-chloro-4-fluorophenyl)amino]-6-({4-[N,N-bis-(2-methoxy-ethyl)-amino]-1-oxo-2-buten-1-yl}amino)-7-[(tetrahydrofuran-2-yl)methoxy]-quinazoline,
(1.30) 4-[(3-ethynyl-phenyl)amino]-6-{[4-(5.5-dimethyl-2-oxo-morpholin-4-yl)-1-oxo-2-buten-1-yl]amino}-quinazoline,
(1.31) 4-[(3-chloro-4-fluoro-phenyl)amino]-6-[2-(2,2-dimethyl-6-oxo-morpholin-4-yl)-ethoxy]-7-methoxy-quinazoline,
(1.32) 4-[(3-chloro-4-fluoro-phenyl)amino]-6-[2-(2,2-dimethyl-6-oxo-morpholin-4-yl)-ethoxy]-7-[(R)-(tetrahydrofuran-2-yl)methoxy]-quinazoline,
(1.33) 4-[(3-chloro-4-fluoro-phenyl)amino]-7-[2-(2,2-dimethyl-6-oxo-morpholin-4-yl)-ethoxy]-6-[(S)-(tetrahydrofuran-2-yl)methoxy]-quinazoline,
(1.34) 4-[(3-chloro-4-fluoro-phenyl)amino]-6-{2-[4-(2-oxo-morpholin-4-yl)-piperidin-1-yl]-ethoxy}-7-methoxy-quinazoline,
(1.35) 4-[(3-chloro-4-fluoro-phenyl)amino]-6-[1-(tert-butyloxycarbonyl)-piperidin-4-yloxy]-7-methoxy-quinazoline,
(1.36) 4-[(3-chloro-4-fluoro-phenyl)amino]-6-(trans-4-amino-cyclohexan-1-yloxy)-7-methoxy-quinazoline,
(1.37) ⁴-[(³-chloro-⁴-fluoro-phenyl)amino]-6-(trans-4-methanesulphonylamino-cyclohexan-1-yloxy)-7-methoxy-quinazoline,
(1.38) 4-[(3-chloro-4-fluoro-phenyl)amino]-6-(tetrahydropyran-3-yloxy)-7-methoxy-quinazoline,
(1.39) 4-[(3-chloro-4-fluoro-phenyl)amino]-6-(1-methyl-piperidin-4-yloxy)-7-methoxy-quinazoline,
(1.40) 4-[(3-chloro-4-fluoro-phenyl)amino]-6-{1-[(morpholin-4-yl)carbonyl]-piperidin-4-yloxy}-7-methoxy-quinazoline,
(1.41) 4-[(3-chloro-4-fluoro-phenyl)amino]-6-{1-[(methoxymethyl)carbonyl]-piperidin-4-yloxy}-7-methoxy-quinazoline,
(1.42) 4-[(3-chloro-4-fluoro-phenyl)amino]-6-(piperidin-3-yloxy)-7-methoxy-quinazoline,
(1.43) 4-[(3-chloro-4-fluoro-phenyl)amino]-6-[1-(2-acetylamino-ethyl)-piperidin-4-yloxy]-7-methoxy-quinazoline,
(1.44) 4-[(3-chloro-4-fluoro-phenyl)amino]-6-(tetrahydropyran-4-yloxy)-7-ethoxy-quinazoline,
(1.45) 4-[(3-chloro-4-fluoro-phenyl)amino]-6-((S)-tetrahydrofuran-3-yloxy)-7-hydroxy-quinazoline,
(1.46) 4-[(3-chloro-4-fluoro-phenyl)amino]-6-(tetrahydropyran-4-yloxy)-7-(2-methoxy-ethoxy)-quinazoline,
(1.47) 4-[(3-chloro-4-fluoro-phenyl)amino]-6-{trans-4-[(dimethylamino)sulphonylamino]-cyclohexan-1-yloxy}-7-methoxy-quinazoline,
(1.48) 4-[(3-chloro-4-fluoro-phenyl)amino]-6-{trans-4-[(morpholin-4-yl)carbonylamino]-cyclohexan-1-yloxy}-7-methoxy-quinazoline,
(1.49) 4-[(3-chloro-4-fluoro-phenyl)amino]-6-{trans-4-[(morpholin-4-yl)sulphonylamino]-cyclohexan-1-yloxy}-7-methoxy-quinazoline,
(1.50) 4-[(3-chloro-4-fluoro-phenyl)amino]-6-(tetrahydropyran-4-yloxy)-7-(2-acetylamino-ethoxy)-quinazoline,
(1.51) 4-[(3-chloro-4-fluoro-phenyl)amino]-6-(tetrahydropyran-4-yloxy)-7-(2-methanesulphonylamino-ethoxy)-quinazoline,
(1.52) 4-[(3-chloro-4-fluoro-phenyl)amino]-6-{l -[(piperidin-1-yl)carbonyl]-piperidin-4-yloxy}-7-methoxy-quinazoline,
(1.53) 4-[(3-chloro-4-fluoro-phenyl)amino]-6-)1-aminocarbonylmethyl-piperidin-4-yl-oxy)-7-methoxy-quinazoline,
(1.54) 4-[(3-chloro-4-fluoro-phenyl)amino]-6-(cis-4-{N-[(tetrahydropyran-4-yl)carbonyl]-N-methyl-amino}-cyclohexan-1-yloxy)-7-methoxy-quinazoline,
(1.55) 4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline,
(1.56) 4-[(3-chloro-4-fluoro-phenyl)amino]-6-(cis-4-{N-[(morpholin-4-yl)sulphonyl]-N-methyl-amino}-cyclohexan-1-yloxy)-7-methoxy-quinazoline,
(1.57) 4-[(3-chloro-4-fluoro-phenyl)amino]-6-(trans-4-ethansulphonylamino-cyclohexan-1-yloxy)-7-methoxy-quinazoline,
(1.58) 4-[(3-chloro-4-fluoro-phenyl)amino]-6-)1-methanesulphonyl-piperidin-4-yloxy)-7-ethoxy-quinazoline,
(1.59) 4-[(3-chloro-4-fluoro-phenyl)amino]-6-)1-methanesulphonyl-piperidin-4-yloxy)-7-(2-methoxy-ethoxy)-quinazoline,
(1.60) 4-[(3-chloro-4-fluoro-phenyl)amino]-6-[l-(2-methoxy-acetyl)-piperidin-4-yloxy]-7-(2-methoxy-ethoxy)-quinazoline,
(1.61) 4-[(3-chloro-4-fluoro-phenyl)amino]-6-(cis-4-acetylamino-cyclohexan-1-yloxy)-7-methoxy-quinazoline,
(1.62) 4-[(3-ethynyl-phenyl)amino]-6-[l -(tert.-butyloxycarbonyl)-piperidin-4-yloxy]-7-methoxy-quinazoline,
(1.63) 4-[(3-ethynyl-phenyl)amino]-6-(tetrahydropyran-4-yloxy]-7-methoxy-quinazoline,
(1.64) 4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline,
(1.65) 4-[(3-chloro-4-fluoro-phenyl)amino]-6-(cis-4-{N-[(4-methyl-piperazin-1-yl)-carbonyl]-N-methyl-amino}-cyclohexan-1-yloxy)-7-methoxy-quinazoline,
(1.66) 4-[(3-chloro-4-fluoro-phenyl)amino]-6-{cis-4-[(morpholin-4-yl)carbonylamino]-cyclohexan-1-yloxy}-7-methoxy-quinazoline,
(1.67) 4-[(3-chloro-4-fluoro-phenyl)amino]-6-{l -[2-(2-oxopyrrolidin-1-yl)ethyl]-piperidin-4-yloxy}-7-methoxy-quinazoline,
(1.68) 4-[(3-chloro-4-fluoro-phenyl)amino]-6-{l -[(morpholin-4-yl)carbonyl]-piperidin-4-yloxy}-7-(2-methoxy-ethoxy)-quinazoline,
(1.69) 4-[(3-ethynyl-phenyl)amino]-6-)1-acetyl-piperidin-4-yloxy)-7-methoxy-quinazoline,
(1.70) 4-[(3-ethynyl-phenyl)amino]-6-)1-methyl-piperidin-4-yloxy)-7-methoxy-quinazoline,
(1.71) 4-[(3-ethynyl-phenyl)amino]-6-)1-methanesulphonyl-piperidin-4-yloxy)-7-methoxy-quinazoline,
(1.72) 4-[(3-chloro-4-fluoro-phenyl)amino]-6-)1-methyl-piperidin-4-yloxy)-7(2-methoxy-ethoxy)-quinazoline,
(1.73) 4-[(3-chloro-4-fluoro-phenyl)amino]-6-)1-isopropyloxycarbonyl-piperidin-4-yloxy)-7-methoxy-quinazoline,
(1.74) 4-[(3-chloro-4-fluoro-phenyl)amino]-6-(cis-4-methylamino-cyclohexan-1-yloxy)-7-methoxy-quinazoline,
(1.75) 4-[(3-chloro-4-fluoro-phenyl)amino]-6-{cis-4-[N-(2-methoxy-acetyl)-N-methyl-amino]-cyclohexan-1-yloxy}-7-methoxy-quinazoline,
(1.76) 4-[(3-ethynyl-phenyl)amino]-6-(piperidin-4-yloxy)-7-methoxy-quinazoline,
(1.77) 4-[(3-ethynyl-phenyl)amino]-6-[l -(2-methoxy-acetyl)-piperidin-4-yloxy]-7-methoxy-quinazoline,
(1.78) 4-[(3-ethynyl-phenyl)amino]-6-{l -[(morpholin-4-yl)carbonyl]-piperidin-4-yloxy}-7-methoxy-quinazoline,
(1.79) 4-[(3-chloro-4-fluoro-phenyl)amino]-6-{l -[(cis-2.6-dimethyl-morpholin-4-yl)carbonyl]-piperidin-4-yloxy}-7-methoxy-quinazoline,
(1.80) 4-[(3-chloro-4-fluoro-phenyl)amino]-6-{l -[(2-methyl-morpholin-4-yl)carbonyl]-piperidin-4-yloxy}-7-methoxy-quinazoline,
(1.81) 4-[(3-chloro-4-fluoro-phenyl)amino]-6-{l -[(S,S)-(2-oxa-5-aza-bicyclo[2,2,1]hept-5-yl)carbonyl]-piperidin-4-yloxy}-7-methoxy-quinazoline,
(1.82) 4-[(3-chloro-4-fluoro-phenyl)amino]-6-{l -[(N-methyl-N-2-methoxyethyl-amino)-carbonyl]-piperidin-4-yloxy}-7-methoxy-quinazoline,
(1.83) 4-[(3-chloro-4-fluoro-phenyl)amino]-6-)1-ethyl-piperidin-4-yloxy)-7-methoxy-quinazoline,
(1.84) 4-[(3-chloro-4-fluoro-phenyl)amino]-6-{l -[(2-methoxyethyl)carbonyl]-piperidin-4-yloxy}-7-methoxy-quinazoline,
(1.85) 4-[(3-chloro-4-fluoro-phenyl)amino]-6-{l-[(3-methoxypropyl-amino)-carbonyl]-piperidin-4-yloxy}-7-methoxy-quinazoline,
(1.86) 4-[(3-chloro-4-fluoro-phenyl)amino]-6-[cis-4-(N-methanesulphonyl-N-methyl-amino)-cyclohexan-1-yloxy]-7-methoxy-quinazoline,
(1.87) 4-[(3-chloro-4-fluoro-phenyl)amino]-6-[cis-4-(N-acetyl-N-methyl-amino)-cyclohexan-1-yloxy]-7-methoxy-quinazoline,
(1.88) 4-[(3-chloro-4-fluoro-phenyl)amino]-6-(trans-4-methylamino-cyclohexan-1-yloxy)-7-methoxy-quinazoline,
(1.89) 4-[(3-chloro-4-fluoro-phenyl)amino]-6-[trans-4-(N-methanesulphonyl-N-methyl-amino)-cyclohexan-1-yloxy]-7-methoxy-quinazoline,
(1.90) 4-[(3-chloro-4-fluoro-phenyl)amino]-6-(trans-4-dimethylamino-cyclohexan-1-yloxy)-7-methoxy-quinazoline,
(1.91) 4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline,
(1.92) 4-[(3-chloro-4-fluoro-phenyl)amino]-6-{1-[2-(2-oxo-3-methyl-imidazolidin-1-yl)-ethyl]-piperidin-4-yloxy}-7-methoxy-quinazoline,
(1.93) 4-[(3-chloro-4-fluoro-phenyl)amino]-6-{1-[2-(2-oxo-hexahydropyrimidin-1-yl)-ethyl]-piperidin-4-yloxy}-7-methoxy-quinazoline,
(1.94) 4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline,
(1.95) 4-[(3-chloro-4-fluoro-phenyl)amino]-6-(1-methanesulphonyl-piperidin-4-yloxy)-7-methoxy-quinazoline,
(1.96) 4-[(3-chloro-4-fluoro-phenyl)amino]-6-(1-cyano-piperidin-4-yloxy)-7-methoxy-quinazoline,
(1.97) 4-[(3-chloro-4-fluoro-phenyl)amino]-6-(tetrahydropyran-4-yloxy)-7-methoxy-quinazoline,
(1.98) 4-[(3-chloro-4-fluoro-phenyl)amino]-6-(1-methylcarbonyl-piperidin-4-yloxy)-7-methoxy-quinazoline,
(1.99) 4-[(3-chloro-4-fluoro-phenyl)amino]-6-(1-dimethylaminoacetyl-piperidin-4-yloxy)-7-methoxy-quinazoline,
(1.100) 4-[(3-chloro-4-fluoro-phenyl)amino]-6-{1-[(dimethylamino)carbonylmethyl]-piperidin-4-yloxy}-7-methoxy-quinazoline, and
(1.101) 4-[(3-chloro-4-fluoro-phenyl)amino]-6-(1-methanesulphonyl-piperidin-4-yloxy)-quinazoline,
and the tautomers and pharmaceutically acceptable salts thereof.

2. The method according to claim 1 wherein the compound employed is selected from the group consisting of:
4-[(3-chloro-4-fluorophenyl)amino]-6-{[4-(morpholin-4-yl)-1-oxo-2-buten-1-yl]amino}-7-cyclopropylmethoxy-quinazoline;
4-[(R)-(1-phenyl-ethyl)amino]-6-{[4-(morpholin-4-yl)-1-oxo-2-buten-1-yl]amino}-7-cyclopentyloxy-quinazoline;
4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline;
4-[(3-chloro-4-fluoro-phenyl)amino]-6-[2-((S)-6-methyl-2-oxo-morpholin-4-yl)-ethoxy]-7-methoxy-quinazoline;
4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline;
4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline;
4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline;
4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline;
4-[(R)-(1-phenyl-ethyl)amino]-6-(4-hydroxy-phenyl)-7H-pyrrolo[2.3-d]pyrimidine; -cyano-4-[(3-chloro-4-fluorophenyl)amino]-6-{[4-(N,N-dimethylamino)-1-oxo-2-buten-1-yl]amino}-7-ethoxy-quinoline;
4-[(R)-(1-phenyl-ethyl)amino]-6-{[4-((R)-6-methyl-2-oxo-morpholin-4-yl)-1-oxo-2-buten-1-yl]amino}-7-methoxy-quinazoline;
4-[(3-chloro-4-fluorophenyl)amino]-6-{[4-(morpholin-4-yl)-1-oxo-2-buten-1-yl]amino}-7-[(tetrahydrofuran-2-yl)methoxy]-quinazoline;
4-[(3-ethynyl-phenyl)amino]-6-{[4-(5.5-dimethyl-2-oxo-morpholin-4-yl)-1-oxo-2-buten-1-yl]amino}-quinazoline;
4-[(3-chloro-4-fluoro-phenyl)amino]-6-{2-[4-(2-oxo-morpholin-4-yl)-piperidin-1-yl]-ethoxy}-7-methoxy-quinazoline;
4-[(3-chloro-4-fluoro-phenyl)amino]-6-[l -(tert.-butyloxycarbonyl)-piperidin-4-yloxy]-7-methoxy-quinazoline;
4-[(3-chloro-4-fluoro-phenyl)amino]-6-(trans-4-methanesulphonylamino-cyclohexan-1-yloxy)-7-methoxy-quinazoline;
4-[(3-chloro-4-fluoro-phenyl)amino]-6-(tetrahydropyran-3-yloxy)-7-methoxy-quinazoline;
4-[(3-chloro-4-fluoro-phenyl)amino]-6-{1-[(morpholin-4-yl)carbonyl]-piperidin-4-yloxy}-7-methoxy-quinazoline;
4-[(3-chloro-4-fluoro-phenyl)amino]-6-(piperidin-3-yloxy)-7-methoxy-quinazoline;
4-[(3-chloro-4-fluoro-phenyl)amino]-6-[1-(2-acetylamino-ethyl)-piperidin-4-yloxy]-7-methoxy-quinazoline;
4-[(3-chloro-4-fluoro-phenyl)amino]-6-(tetrahydropyran-4-yloxy)-7-ethoxy-quinazoline;
4-[(3-chloro-4-fluoro-phenyl)amino]-6-{trans-4-[(morpholin-4-yl)carbonylamino]-cyclohexan-1-yloxy}-7-methoxy-quinazoline;
4-[(3-chloro-4-fluoro-phenyl)amino]-6-{1-[(piperidin-1-yl)carbonyl]-piperidin-4-yloxy}-7-methoxy-quinazoline;
4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline;
4-[(3-chloro-4-fluoro-phenyl)amino]-6-(trans-4-ethansulphonylamino-cyclohexan-1-yloxy)-7-methoxy-quinazoline;
4-[(3-chloro-4-fluoro-phenyl)amino]-6-(1-methanesulphonyl-piperidin-4-yloxy)-7-(2-methoxy-ethoxy)-quinazoline;
4-[(3-chloro-4-fluoro-phenyl)amino]-6-[1-(2-methoxy-acetyl)-piperidin-4-yloxy]-7-(2-methoxy-ethoxy)-quinazoline;
4-[(3-ethynyl-phenyl)amino]-6-(tetrahydropyran-4-yloxy]-7-methoxy-quinazoline;
4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline;
4-[(3-chloro-4-fluoro-phenyl)amino]-6-{cis-4-[(morpholin-4-yl)carbonylamino]-cyclohexan-1-yloxy}-7-methoxy-quinazoline;
4-[(3-chloro-4-fluoro-phenyl)amino]-6-{1-[2-(2-oxopyrrolidin-1-yl)ethyl]-piperidin-4-yloxy)-7-methoxy-quinazoline;
4-[(3-ethynyl-phenyl)amino]-6-(1-acetyl-piperidin-4-yloxy)-7-methoxy-quinazoline;
4-[(3-ethynyl-phenyl)amino]-6-(1-methyl-piperidin-4-yloxy)-7-methoxy-quinazoline;
4-[(3-ethynyl-phenyl)amino]-6-(1-methanesulphonyl-piperidin-4-yloxy)-7-methoxy-quinazoline;
4-[(3-chloro-4-fluoro-phenyl)amino]-6-(1-methyl-piperidin-4-yloxy)-7(2-methoxy-ethoxy)-quinazoline;
4-[(3-ethynyl-phenyl)amino]-6-{1-[(morpholin-4-yl)carbonyl]-piperidin-4-yloxy}-7-methoxy-quinazoline;
4-[(3-chloro-4-fluoro-phenyl)amino]-6-{1-[(N-methyl-N-2-methoxyethyl-amino)carbonyl]-piperidin-4-yloxy}-7-methoxy-quinazoline;
4-[(3-chloro-4-fluoro-phenyl)amino]-6-(1-ethyl-piperidin-4-yloxy)-7-methoxy-quinazoline;
4-[(3-chloro-4-fluoro-phenyl)amino]-6-{1-[(2-methoxyethyl)carbonyl]-piperidin-4-yloxy}-7-methoxy-quinazoline;
4-[(3-chloro-4-fluoro-phenyl)amino]-6-(trans-4-methylamino-cyclohexan-1-yloxy)-7-methoxy-quinazoline;
4-[(3-chloro-4-fluoro-phenyl)amino]-6-(trans-4-di methylamino-cyclohexan-1-yloxy)-7-methoxy-quinazoline;
4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline;
4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline; and
4-[(3-chloro-4-fluoro-phenyl)amino]-6-)1-methanesulphonyl-piperidin-4-yloxy)-7-methoxy-quinazoline;
and the tautomers and pharmaceutically acceptable salts thereof.

3. The method according to claim 1, wherein the condition to be treated is chronic rhinosinusitis.

4. The method according to claim 1, wherein the condition to be treated is nasal polyposis.

5. The method according to claim 1, wherein the condition to be treated is chronic rhinosinusitis with nasal polyposis.

6. A pharmaceutical composition in the form of a powder for nasal administration containing at least one of the compounds mentioned in claim 1 with an average particle size in the range between about 0.1 and 200 μm and one or more carriers or excipients.

7. A pharmaceutical composition for nasal administration in the form of a suspension containing at least one of the compounds mentioned in claim 1, and a suspension agent.

8. A pharmaceutical composition for nasal administration in the form of a solution containing at least one of the compounds mentioned in claim 1, and at least one solvent.
